(19) United States
(12) Patent Application Publication
(10) Pub. No.: US 2024/0180937 A1
(43) Pub. Date: Jun. 6, 2024

(54) CYTOKINE PRODUCTION SUPPRESSING COMPOSITION CONTAINING D-ALLOSE AS ACTIVE COMPONENT, AND METHOD OF TREATING OR PREVENTING DISEASE ASSOCIATED WITH CYTOKINE OVERPRODUCTION USING SAME

(71) Applicants: NATIONAL UNIVERSITY CORPORATION KAGAWA UNIVERSITY, Takamatsu-shi, Kagawa (JP); SETOLAS HOLDINGS, INC., Takamatsu-shi, Kagawa (JP)

(72) Inventors: Katsuaki Hoshino, Kagawa (JP); Kenjiro Takao, Kagawa (JP); Ken Izumori, Kagawa (JP)

(73) Assignees: NATIONAL UNIVERSITY CORPORATION KAGAWA UNIVERSITY, Takamatsu-shi, Kagawa (JP); SETOLAS HOLDINGS, INC., Takamatsu-shi, Kagawa (JP)

(21) Appl. No.: 18/284,991

(22) PCT Filed: Mar. 4, 2022
(86) PCT No.: PCT/JP2022/009524

§ 371 Date: Sep. 29, 2023

(30) Foreign Application Priority Data
Mar. 30, 2021 (JP) 2021-057101

(51) Int. Cl. A61K 31/7004; A23L 33/125; A61P 37/02

(52) U.S. Cl. CPC A61K 31/7004; A23L 33/125; A61P 37/02

(57) ABSTRACT

The present invention provides a cytokine production suppressing composition containing D-allose as an active component. The present invention further provides a method of treating or preventing diseases associated with cytokine overproduction in a subject, said method involving dosing the subject with the cytokine production suppressing composition containing D-allose as an active component.

### FIELD

The present invention relates to a cytokine production suppressing composition containing D-allose as the active ingredient, and to a method of treating or preventing disease associated with cytokine overproduction using it.

### BACKGROUND

Dendritic cells are largely subclassified as conventional dendritic cells (CDC) or plasmacytoid dendritic cells (pDC), based on their locations in the body and differences in marker molecules expressed on their cell surfaces. Plasmacytoid dendritic cells express nucleic acid-recognizing toll-like receptor 7 (TLR7) and toll-like receptor 9 (TLR9) on their endosomal membranes, and when activated by nucleic acids of viruses or microorganisms, or host nucleic acids, they exhibit very high production of type I interferon (IFN), in an amount of about 1000 times that of conventional dendritic cells.

Type I interferon produced in large amounts by plasmacytoid dendritic cells functions to augment biological defense reaction during viral infection. In autoimmune diseases such as systemic lupus erythematosus (SLE) or psoriasis vulgaris, on the other hand, complexes comprising patient-derived nucleic acids (such as single-stranded RNA or double-stranded DNA) and proteins (such as anti-nucleic acid antibodies or antimicrobial peptides) activate plasmacytoid dendritic cells, inducing high production of type I interferon. The type I interferon that is produced elicits activation of immunocytes (accelerating production of autoantibodies (including anti-nucleic acid antibodies) by B cells, for example), which is thought to further lead to reactivation of plasmacytoid dendritic cells. In other words, it is thought that production of type I interferon by plasmacytoid dendritic cells occurring in autoimmune disease leads to creation of a vicious cycle which exacerbates pathology in the autoimmune disease. It is therefore believed that this vicious cycle can be interrupted by suppressing production of type I interferon by plasmacytoid dendritic cells.

Production of type I interferon by plasmacytoid dendritic cells requires the serine/threonine kinase, IκB kinase a (NPL 1). Results of comparative DNA microarray analysis of gene expression in mice dendritic cell subpopulations have demonstrated that the Ets family transcription factor Spi-B is involved in activation of the type I interferon gene (NPL 2). Other factors including signaling molecules and transcription factors have also been reported to be implicated in production of the type I interferon gene. Drugs that inhibit the functions of these molecules are considered to be promising candidates as therapeutic agents to suppress type I interferon production, but as of the current time there has been no implementation of drugs that regulate production of type I interferon by plasmacytoid dendritic cells.

The term “rare sugar” is defined as a monosaccharide or its derivative having low abundance in the natural world, and about 50 types of rare sugars are known, including D-allulose (D-psicose). In the fields of health and medicine, rare sugars have been confirmed to exhibit effects of suppressing increase in postprandial blood glucose, inhibiting fat accumulation and preventing arteriosclerosis. They are also increasingly being applied for treatment of neurodegenerative diseases, cerebral and myocardial infarction and hypertension, due to their antioxidant effects (PTL 1). D-allulose has also been reported to function as a suppressor against proliferation of cancer cells (PTL 2). However, no reports yet exist regarding the ability of rare sugars to suppress overproduction of cytokines.

### CITATION LIST

### Patent Literature

[PTL 1] International Patent Publication No. WO2003/097820
[PTL 2] International Patent Publication No. WO2016/152293

### Non Patent Literature

[NPL 1] Hoshino K., et al. Nature. 2006 Apr. 13; 440(7086):949-953.
[NPL 2] Sasaki I., et al. Blood. 2012 Dec. 6; 120(24):4733-4743.

### SUMMARY

### Technical Problem

It is an object of the present invention to provide a composition that is able to suppress cytokine production, and a method of treating or preventing disease associated with cytokine overproduction using the composition.

### Solution to Problem

As a result of much ardent research, the present inventors have devised this invention upon finding that the rare sugar D-allose has an effect of suppressing production of cytokines. Specifically, the present invention encompasses the following inventions.
[1] A cytokine production suppressing composition containing D-allose as the active ingredient.
[2] The composition according to [1] above, which suppresses production of a cytokine by plasmacytoid dendritic cells.
[3] The composition according to [1] or [2] above, wherein the cytokine is type I interferon.
[4] The composition according to any one of [1] to [3] above, wherein the D-allose is D-allose and/or a derivative thereof, and/or a mixture of the same.
[5] The composition according to [4] above, wherein the D-allose derivative is one or more D-allose derivatives selected from the group consisting of sugar alcohols in which a carbonyl group or the D-allose is an alcohol group; uronic acids in which an alcohol group of the D-allose is oxidized; amino sugars in which an alcohol group of the D-allose is replaced with an amino group; and D-allose derivatives in which any hydroxyl group of the D-allose is replaced with a hydrogen atom, halogen atom or an amino, carboxyl, nitro, cyano, lower alkyl, lower alkoxy, lower alkanoyl, lower alkanoyloxy, lower alkoxycarbonyl, mono- or di-lower alkyl substituted amino, aralkyl, aryl or heteroaryl group.
[6] The composition according to any one of [1] to [5] above, which is for treatment or prevention of a disease associated with cytokine overproduction.
[7] The composition according to any one of [1] to [5] above, wherein the disease associated with cytokine overproduction is selected from the group consisting of collagen disease, joint or muscular inflammation or pain (such as chronic rheumatoid arthritis, rheumatoid spondylitis, osteoarthritis or uric acid arthritis), skin inflammation (such as eczema), systemic lupus erythematosus, inflammatory chronic kidney conditions (such as glomerular nephritis, lupus nephritis or membranous nephritis), Sjogren's syndrome, and psoriasis.
[8] The composition according to any one of [1] to [7] above, which is a drug.
[9] The composition according to any one of [1] to [7] above, which is a food.
[10] The composition according to [9] above, wherein the food is a health functional food or dietary supplement.
[11] The composition according to above, wherein the health functional food is a specified health food or nutritional function food.
[12] A method for treatment or prevention of a disease associated with cytokine overproduction in a subject, comprising:
administering a cytokine production suppressing composition containing D-allose as the active ingredient, to the subject in need thereof.
[13] The method according to [12] above, wherein the disease associated with cytokine overproduction is selected from the group consisting of collagen disease, joint or muscular inflammation or pain (such as chronic rheumatoid arthritis, rheumatoid spondylitis, osteoarthritis or uric acid arthritis), skin inflammation (such as eczema), systemic lupus erythematosus, inflammatory chronic kidney conditions (such as glomerular nephritis, lupus nephritis or membranous nephritis), Sjogren's syndrome, and psoriasis.
[14] The method according to [12] or [13] above, which suppresses production of cytokines by plasmacytoid dendritic cells.
[15] The method according to any one of [12] to [14] above, wherein the cytokine is type I interferon.
[16] The method according to any one of [12] to [15] above, wherein the D-allose is D-allose and/or a derivative thereof, and/or a mixture of the same.
[17] The method according to [16] above, wherein the D-allose derivative is one or more D-allose derivatives selected from the group consisting of sugar alcohols in which a carbonyl group of the D-allose is an alcohol group; uronic acids in which an alcohol group of the D-allose is oxidized; amino sugars in which an alcohol group of the D-allose is replaced with an amino group; and D-allose derivatives in which any hydroxyl group of the D-allose is replaced with a hydrogen atom, halogen atom or an amino, carboxyl, nitro, cyano, lower alkyl, lower alkoxy, lower alkanoyl, lower alkanoyloxy, lower alkoxycarbonyl, mono- or di-lower alkyl substituted amino, aralkyl, aryl or heteroaryl group.
[18] The method according to any one of [12] to [16] above, wherein the composition is administered as a drug.
[19] The method according to any one of [12] to [16] above, wherein the composition is administered as a food.
[20] The method according to [19] above, wherein the food is a health functional food or dietary supplement.
[21] The method according to [20] above, wherein the health functional food is a specified health food or nutritional function food.
[22] Use of D-allose for production of a composition for treatment or prevention of a disease associated with cytokine overproduction.

### Advantageous Effects of Invention

The present invention can be used for treatment or prevention of diseases related to overproduction of cytokines, such as overproduction of type I interferon by plasmacytoid dendritic cells. The present invention can also provide a novel therapeutic agent and treatment method that can interrupt the vicious cycle that exacerbates pathology in diseases where overproduction of cytokines is one of the causes, such as autoimmune diseases.

### BRIEF DESCRIPTION OF DRAWINGS

FIG. 1 shows the results of flow cytometry analysis of dendritic cells isolated from mouse spleen and bone marrow. Shown are dendritic cells isolated from (A) spleen or (B) bone marrow (pDC=plasmacytoid dendritic cells, cDC=conventional dendritic cells).

FIG. 2 shows the results of comparing cytokine production by mouse plasmacytoid dendritic cells (pDC) with and without a rare sugar (D-allose). Shown are the results of comparing cytokine production by (A) spleen-derived plasmacytoid dendritic cells (pDC) and (B) bone marrow-derived plasmacytoid dendritic cells (pDC).

FIG. 3 shows the results of comparing cytokine production by mouse spleen-derived conventional dendritic cells (cDC) with and without a rare sugar (D-allose).

### DESCRIPTION OF EMBODIMENTS

Embodiments for carrying out the invention will be described in detail below, with the understanding that the technical scope of the invention is not limited only to these embodiments. The prior art documents cited herein are incorporated in their entirety by reference throughout the present specification.

According to one embodiment, the invention provides a composition for suppressing production of cytokines, containing D-allose as the active ingredient. The composition of the invention may be provided as a drug or it may be provided as a food. The composition of the invention used as a food may be a health functional food (such as a specified health food or nutritional function food), or a dietary supplement, for example.

According to another embodiment, the invention provides a method for treatment or prevention of a disease associated with cytokine overproduction in a subject, comprising:
administering a cytokine production suppressing composition containing D-allose as the active ingredient, to the subject in need thereof.

The present invention was completed upon finding that D-allose can specifically suppress production of cytokines, and especially production of cytokines by plasmacytoid dendritic cells.

As used herein, the phrase “suppress(ing) production of cytokines” is to be understood in the widest sense of being a biological effect of the composition of the invention whereby production of cytokines is directly and/or indirectly inhibited or significantly suppressed. According to one embodiment, for example, administration of the composition of the invention suppresses production of cytokines, such as type I interferon, by plasmacytoid dendritic cells from a subject with disease, due to overproduction compared to the level of cytokines produced by plasmacytoid dendritic cells in healthy persons, for example. The degree of “suppressing production of cytokines” is not particularly restricted as it will vary depending on the dose and administration period of the composition of the invention and the body weight/body height, gender, age and symptoms of the subject. The method of detecting the degree of “suppressing production of cytokines” is not particularly restricted and may be a method of measuring cytokine levels, or it may be a method of comparison by measuring expression levels of a particular cytokine-producing gene by a publicly known method.

Plasmacytoid dendritic cells are classified as a type of dendritic cells (DCs). Dendritic cells are potent antigen-presenting cells. They are present in the blood, tissues and lymphatic organs and phagocytose foreign substances such as microbes and cancer cells, expressing the antigenic peptides on Major Histocompatibility Complex (MHC) Class I and Class II molecules displayed by DCs, and thereby activating CD4 T cells and CD8 T cells, respectively. This elicits an immune response in the body specific to the antigen, leading to elimination of the foreign substances.

Dendritic cells are largely subclassified into conventional dendritic cells (cDC) and plasmacytoid dendritic cells (pDC). Plasmacytoid dendritic cells express nucleic acid-recognizing toll-like receptor 7 (TLR7) and toll-like receptor 9 (TLR9) on their endosomal membranes, and when activated by nucleic acids of viruses or microorganisms, or host nucleic acids, they exhibit very high production of type I interferon (IFN). Overproduction of cytokines such as type I interferon by plasmacytoid dendritic cells leads to a vicious cycle that exacerbates pathology in autoimmune disease. According to one embodiment, therefore, the invention can suppress production of cytokines, such as the production of cytokines, and especially type I interferon (IFN), by plasmacytoid dendritic cells. The invention can therefore contribute to treatment or prevention of diseases associated with overproduction of cytokines. According to one embodiment, the composition of the invention can suppress cytokine production from plasmacytoid dendritic cells by TLR7 ligand stimulation, and preferably can suppress cytokine production from plasmacytoid dendritic cells by stimulation of TLR7 ligands having an average molecular weight of about 800,000 or greater.

The term “type I interferon” includes interferon-α (IFN-α) and interferon-β (IFN-β), of the interferon family. According to one embodiment, the composition of the invention can suppress production of cytokines such as interferon-α (IFN-α) and/or interferon-β (IFN-β), as type I interferons.

A disease associated with overproduction of cytokines may be one selected from the group consisting of collagen disease, joint or muscular inflammation or pain (such as chronic rheumatoid arthritis, rheumatoid spondylitis, osteoarthritis or uric acid arthritis), skin inflammation (such as eczema), systemic lupus erythematosus, inflammatory chronic kidney conditions (such as glomerular nephritis, lupus nephritis or membranous nephritis), Sjogren's syndrome, and psoriasis, for example.

The D-allose to be used for the invention is in extremely low abundance in the natural world, compared to D-glucose (glucose) which is abundantly present. Of the monosaccharides that are the base units of sugars (a total of 34 monosaccharides with 6 carbon atoms (hexoses) exist, 16 of which are aldoses, 8 of which are ketoses and 10 of which are sugar alcohols), monosaccharides that are only present in trace amounts in the natural world (aldoses and ketoses) and their derivatives (sugar alcohols) are defined as “rare sugars”, in contrast to “naturally-occurring monosaccharides”, typically D-glucose (glucose), that are highly abundant in the natural world. The rare sugars that can be mass produced as of the current writing are D-psicose and D-allose. D-allose is the D-form of allose, classified as an aldose, and it is a hexose.

Previously disclosed methods for obtaining “D-allose” include a method of synthesis from D-psicose using L-rhamnose isomerase, and a method of reacting D-xylose isomerase with a D-psicose-containing solution, but the D-allose of the invention is not limited to these methods and may be obtained by any method of isomerization by chemical treatment. The D-psicose used as starting material for D-allose will usually be obtained by treating fructose with an enzyme (epimerase), but this is not limitative, and it can be obtained by a method using microorganisms that produce the enzyme, or it may be an extract from a natural product, or a substance that is present in a natural product, or a substance isomerized by a chemical treatment method. The method for purifying the D-psicose using an enzyme may be a publicly known method.

The D-allose may also be in the form of D-allose-containing syrup. A D-allose-containing syrup can be obtained by appropriately mixing common syrups (liquid sugar), but it is also sold in stores as a “food” such as the commercial product “Rare Sugar Sweet” (manufacturer: RareSweet Co., Ltd., vendor: Matsutani Chemical Industry Co., Ltd.), and is readily available.

The method of obtaining the D-allose-containing syrup may be, for example, reacting an alkali with a monosaccharide (D-glucose or D-fructose) to cause Lobry de Bruyn-Van Ekenstein transformation or retro-aldol reaction and subsequent aldol reaction (such reactions known as “alkali isomerization reaction”). The syrup containing the monosaccharides (including rare sugars) produced thereby can be generally referred to as “rare sugar-containing syrup”, and one example thereof is a syrup made from D-glucose and/or D-fructose as the starting material and an alkali-isomerized to a D-glucose and/or D-fructose content of 55 to 99 mass %. The product “Rare Sugar Sweet” is a syrup containing rare sugars obtained by the method disclosed in International Patent Publication No. WO2010/113785 with isomerized sugar as the starting material, and it was produced so as to contain primarily D-psicose and D-allose as the rare sugars. The rare sugars in the rare sugar-containing syrup obtained by this method are 0.5 to 17 mass % D-psicose and 0.2 to 10 mass % D-allose, as proportions with respect to the total sugars. A publication by Takamine et al. (Ouyou Toushitsu Kagaku (Bulletin of Applied Glycoscience), Vol. 5, No. 1, 44-49(2015)) reports that it is a syrup containing 5.4 g/100 g D-psicose, 5.3 g/100 g D-sorbose, 2.0 g/100 g D-tagatose, 1.4 g/100 g D-allose and 4.3 g/100 g D-mannose.

The starting materials to be used to produce the rare sugar-containing syrup are starch, sugar, isomerized sugar, fructose or glucose, etc. Isomerized sugar is widely considered to be a mixture of sugars with a main composition of D-glucose and D-fructose in a specified compositional ratio, and generally refers to syrup consisting mainly of glucose and fructose, obtained by isomerization with glucose isomerase or alkali isomerization of a sugar solution composed mainly of glucose from hydrolysis of starch using an enzyme such as amylase or an acid. According to the JAS standard, the term “glucose-fructose syrup” is applied to a fructose content (percentage of fructose of the total sugars) of less than 50%, “fructose-glucose syrup” is applied for ≥50% and <90%, “high fructose syrup” is applied for ≥90%, and “sugar-containing fructose-glucose syrup” is applied for syrups with addition of sugar to glucose-fructose syrup in an amount not exceeding the glucose-fructose syrup, and the starting material for the rare sugar-containing syrup of the invention may employ any of these isomerized sugars.

For example, rare sugar-containing syrup obtained using D-fructose as the starting material contains 5.2% D-psicose, 1.8% D-allose, 15.0% glucose and 69.3% D-fructose. Rare sugar-containing syrup obtained using isomerized sugar as the starting material contains 3.7% D-psicose, 1.5% D-allose, 45.9% glucose and 37.7% D-fructose, or when D-glucose is used as the starting material it contains 5.7% D-psicose, 2.7% D-allose, 47.4% glucose and 32.1% D-fructose, although the sugar composition will differ depending on differences in the starting material and treatment method. D-allose separated and purified from such syrups may also be used, or the syrups may be used directly.

According to one aspect, the D-allose to be used for the invention may be D-allose and/or its derivative, and/or a mixture thereof. Compounds whose molecular structure is altered from starting compounds by chemical reaction are generally referred to as derivatives of the starting compounds. Throughout the present specification, “D-allose derivative” is used to mean a compound obtained by converting the molecular structure of the D-allose starting compound by chemical reaction; and also includes compounds obtained by converting the molecular structure of a compound similar to D-allose (such as D-glucose) as the starting compound by chemical reaction, which have the same structure as a compound obtained by converting the molecular structure of D-allose by chemical reaction (also referred to as “D-allose structural analog”). Derivatives of hexoses including D-allose are commonly known, such as sugar alcohols (reduction of monosaccharides converts aldehyde and ketone groups to alcohol groups, producing a polyhydric alcohol with the same number of carbon atoms), uronic acids (monosaccharides with an oxidized alcohol group, of which known natural examples include D-glucuronic acid, galacturonic acid and mannuronic acid), and amino sugars (having an OH group of the sugar molecule replaced with an NH₂ group, e.g., glucosamine, chondrosamine or a glycoside), though with no limitation to these. The D-allose derivative may also be a D-allose derivative selected from among sugar alcohols with the carbonyl group of D-allose converted to an alcohol group, uronic acids with an alcohol group of D-allose oxidized, and amino sugars with an alcohol group of D-allose replaced with an amino group.

According to another aspect, the D-allose derivative may be a D-allose derivative in which any hydroxyl group of D-allose (such as the hydroxyl group at position 2, position 3, position 4, position 5 and/or position 6) is replaced with a hydrogen atom, a halogen atom, an amino, carboxyl, nitro, cyano, lower alkyl, lower alkoxy, lower alkanoyl, lower alkanoyloxy or lower alkoxycarbonyl group, a mono- or di-lower alkyl substituted amino group, or an aralkyl, aryl or heteroaryl group.

Halogen atoms are the atoms fluorine, chlorine, bromine or iodine. The alkyl portion of a lower alkyl, lower alkoxy, lower alkoxycarbonyl or mono- or di-lower alkyl substituted amino group is a straight-chain, branched or cyclic C1-C6 alkyl group, with specific examples including methyl, ethyl, propyl, isopropyl, butyl, isobutyl, sec-butyl, tert-butyl, pentyl, isopentyl, neopentyl, hexyl, isohexyl, cyclopropyl, cyclobutyl, 2-methylcyclopropyl, cyclopropylmethyl, cyclopentyl and cyclohexyl, etc.

The lower alkanoyl portion of a lower alkanoyl or lower alkanoyloxy group is a straight-chain, branched or cyclic C1-C7 alkanoyl group, with specific examples including formyl, acetyl, propionyl, butyryl, isobutyryl, valeryl, isovaleryl, pivaloyl, hexanoyl, cyclopropylcarbonyl, cyclobutylcarbonyl, 2-methylcyclopropylcarbonyl and cyclohexylcarbonyl, etc.

An aralkyl group is a C7-C20 aralkyl group, with specific examples including benzyl, phenethyl, α-methylbenzyl, benzhydril, trityl and naphthylmethyl, etc.

An aryl group is a C6-C14 aryl group, with specific examples including phenyl and naphthyl, etc.

A heteroaryl group is a C3-C8 heteroaryl group, which is a monocyclic, polycyclic or fused ring of 1 to 4 N, O or S atoms which may be the same or different, with specific examples including 2-pyridyl, 3-pyridyl, 4-pyridyl, 2-quinonyl, 3-quinonyl, 4-quinonyl, 5-quinonyl, 6-quinonyl, 7-quinonyl, 8-quinonyl, 2-indolyl, 3-indolyl, 4-indolyl, 5-indolyl, 6-indolyl, 7-indolyl, 2-furyl, 3-furyl, 2-thienyl, 3-thienyl, 2-pyrrolidyl, 3-pyrrolidyl, 2-imidazolyl, 4-imidazolyl, 5-imidazolyl, 2-oxazolyl, 4-oxazolyl, 5-oxazolyl, 2-thiazolyl, 4-thiazolyl and 5-thiazolyl, etc.

According to one aspect, the D-allose derivative to be used for the invention may be a D-allose derivative such as 2-deoxy-D-allose, 5-deoxy-D-allose, 6-deoxy-D-allose or 3-deoxy-D-allose(3-deoxy-D-glucose), etc.

Through the present specification, “D-allose and/or a derivative thereof, and/or a mixture thereof” may also be shortened to simply “D-allose”. The “D-allose and/or a derivative thereof, and/or a mixture thereof” to be used for the invention is also interpreted as including pharmacologically acceptable salts and/or hydrates.

Throughout the present specification, the term “food” means food in general, but in addition to common foods including health foods, it also includes health functional foods such as specified health foods and nutritional function foods, as well as dietary supplements (supplements and nutritional supplements), feeds and food additives. The composition for suppressing production of cytokines of the invention has the food compound D-allose as the active ingredient, and may be used as a sweetener, seasoning, food additive, food material, food or beverage, health food or beverage, pharmaceutical or quasi drug, or feed, being able to suppress production of cytokines in any of these forms.

According to one embodiment, the composition of the invention may be administered by any desired route of administration. Routes of administration include local administration (percutaneous, inhalation, enema, eye drop, ear drop, pernasal or intravaginal), enteral administration (oral, tubal or enteral), or parenteral administration (intravenous, transarterial, transdermal or intramuscular injection).

According to one embodiment, the dosage of D-allose for the invention may be adjusted as appropriate to be a dosage at which the effect of the invention is exhibited, and for example, it may be administered at 1 mg/kg body weight/day to 1000 mg/kg body weight/day, with adjustment as appropriate depending on the age and symptoms. It may be administered at a dosage of 1 mg/kg body weight/day to 1000 mg/kg body weight/day, such as 10 mg/kg body weight/day to 800 mg/kg body weight/day or 50 mg/kg body weight/day to 500 mg/kg body weight/day, as an amount that can be ingested for transintestinal administration.

When the rare sugar D-allose and/or a derivative thereof, and/or a mixture thereof is used as a component of a food, an effective dose of the rare sugar D-allose and/or a derivative thereof, and/or a mixture thereof, can be safely ingested in the course of routine dietary habit. The rare sugar D-allose is an aldose and is therefore a highly safe compound for administration to humans.

Throughout the present specification, the term “drug” is used to include drugs and quasi drugs.

The D-allose and/or a derivative thereof, and/or a mixture thereof in the composition of the invention may include suitable additives such as excipients, stabilizers, preservatives, binders or disintegrators, and may be provided by formulation, selecting a suitable dosage form such as tablets, powder, granules, capsules, a solution, syrup, elixir or an oily or aqueous suspension.

For a solid formulation, the D-allose and/or a derivative thereof, and/or a mixture thereof may include pharmaceutically acceptable additives, and for example, fillers or extenders, binders, disintegrators, dissolution accelerators, moistening agents or lubricants may be selected as necessary for mixing and formulation. An excipient, disintegrator, binder and lubricant may be added to and mixed with the D-allose of the invention and/or a derivative thereof, and/or a mixture thereof, and the mixture may then be compacted and molded. Lactose, starch or mannitol is commonly used as excipients. Calcium carbonate or carboxymethyl cellulose calcium is commonly used as disintegrators. Gum arabic, carboxymethyl cellulose or polyvinylpyrrolidone is used as binders. Talc and magnesium stearate are publicly known lubricants.

Tablets can be coated in a publicly known manner for masking or as an enteric-coated formulation. Ethyl cellulose or polyoxyethylene glycol may be used as coating agents.

In addition to the aforementioned drugs (pharmaceutical compositions), the composition of the invention can also be provided as a food (such as a medical food, specified health food, health assisting food, health food, nutritional function food, supplement, dietary supplement or herbal tea). D-allose may be administered at a dosage of 1 mg/kg body weight/day to 1000 mg/kg body weight/day (for example, 50 mg to 50 g/day for a 50 kg adult).

Subjects for which the invention may be used include animals including humans (mammals such as humans, cows, pigs, dogs and cats, and birds such as chickens).

According to one embodiment, the invention may be used in combination with publicly known therapeutic agents for diseases associated with overproduction of cytokines, such as immunosuppressive agents and immunomodulators, or it may be used in place of such agents. An immunosuppressive agent is a drug that acts in a suppressive manner on the immune system and is used for treatment of an autoimmune disease, such agents including folic acid, antimetabolites, calcineurin inhibitors, corticosteroid agents (also known simply as “steroids”), nucleic acid antimetabolites, nucleic acid synthesis inhibitors, biological preparations targeting cell surface antigens, and biological preparations targeting cytokines or cytokine receptors. Examples include, but are not limited to, the folic acid antimetabolite Methotrexate, the calcineurin inhibitors Cyclosporin and Tacrolimus, the corticosteroid agents Methylprednisolone, Prednisolone and Dexamethasone, the nucleic acid synthesis inhibitors Cyclophosphamide and Azathioprine, the nucleic acid antimetabolite Mycophenolate mofetil, the cell surface antigen-targeting biological preparations Rituximab and Abatacept, and the cytokine- or cytokine receptor-targeting biological preparations Belimumab, Adalimumab, Etanercept and Tocilizumab. An immunomodulator is a drug that normalizes abnormal immunity to cure disease, and an example is the antimalarial drug Hydroxychloroquine.

The present invention will now be explained in greater detail by Examples, with the understanding that the invention is not limited in any way by the Examples.

### EXAMPLES

Dendritic cells (DC) have high antigen-presenting ability and produce cytokines by stimulation of innate immunity, thus playing a role in initiating acquired immunity via activation of T cells. In order to elucidate the function of DCs, the present inventors examined the immune response of DCs using the tool of rare sugars (D-allose, D-allulose or L-allulose), in addition to D-glucose or D-fructose. The goal was to use rare sugars to develop a method for treating diseases by modulating DC function.

### (1) Method

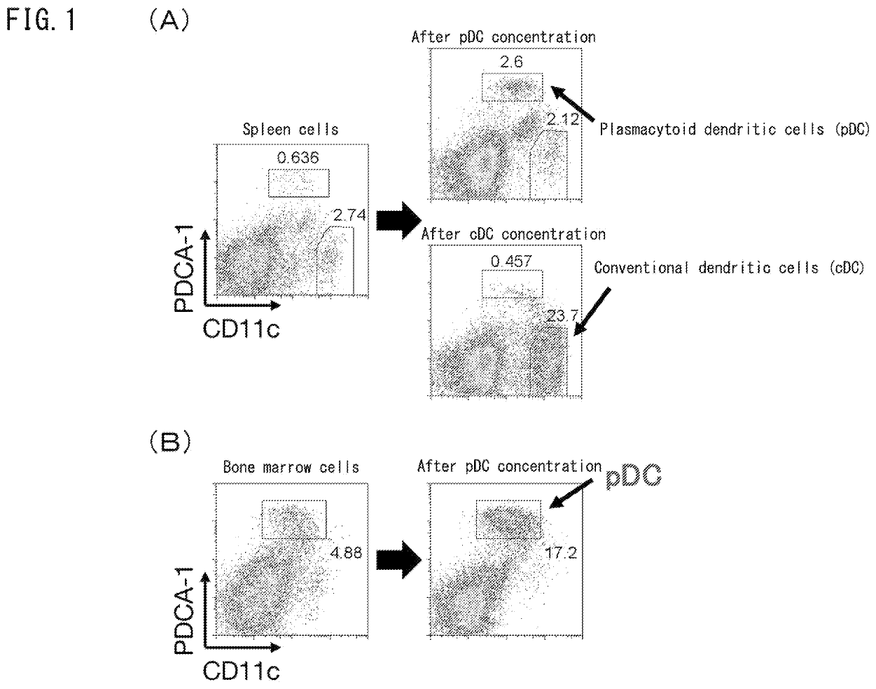

CD317-positive cells were prepared by magnetic bead sorting (MACS) from spleen cells obtained from C57BL/6N mice (Clea Japan, Inc.), as plasmacytoid dendritic cells (pDC) (FIG. 1(A)). CD11c-positive cells in the CD317-negative cell fraction were then prepared using magnetic beads, as conventional dendritic cells (CDC) (FIG. 1(A)). CD317-positive cells were also prepared using magnetic beads from bone marrow cells obtained from C57BL/6N mice (Clea Japan, Inc.), as bone marrow pDCs (FIG. 1(B)).

The obtained pDCs and cDCs were inoculated into a 96-well plate using glucose-free PRMI1640 medium with addition of 10 v/v % FCS, 100 μM 2-mercaptoethanol, 50 μg/ml streptomycin and 50 units/ml penicillin G, after which a rare sugar (D-allose, D-allulose or L-allulose) or D-glucose or D-fructose, was added to a final concentration of 0.2 w/v %.

The pDCs were stimulated with non-stimulating medium and TLR7 ligand (polyuridylic acid at a final concentration of 2.5 μg/ml (Poly(U) (Sigma) mixed with Lipofectamine 2000 (Thermo Fisher Scientific)), 100 nM R848 (InvivoGen), or TLR9 ligand (synthetic DNA (3 μM of D19: 5′-GGtgcatcgatgcagGGGGg-3′ and 0.03 μM of 1668: 5′-TCCATGACGTTCCTGATGCT-3′ (where uppercase indicates phosphorothioate DNA and lowercase indicates phosphodiester DNA))), and the IFN-α in the culture supernatant after 20 to 24 hours was measured using ELISA by InvivoGen while the IL-12p40 was measured using ELISA by Biolegend.

The cDCs were stimulated with non-stimulating medium, 100 nM of R848 and 1 μM of 1668, and the IL-12p40 in the culture supernatant after 20 to 24 hours was measured using ELISA by Biolegend.

After administering 2 ml of 4 w/v % Brewer thioglycollate medium (Sigma) intraperitoneally to C57BL/6N mice, the intraperitoneal macrophages on the 3rd day were collected and used. After inoculation into the same medium as the DCs, stimulation was with 100 ng/ml of Lipopolysaccharide E. coli O111:B4 (Sigma) and 5 ng/ml of IFN-γ (Biolegend), the TNF-α in the culture supernatant after 20 to 24 hours was measured using ELISA by Biolegend and the nitric oxide was measured using a NO2/NO3 Assay Kit-C II (Colorimetric) (Griess Reagent Kit) (Dojindo Laboratories).

### (2) Results

### (2-1) Cytokine Production Accompanying Activation of Plasmacytoid Dendritic Cells is Suppressed by D-Allose

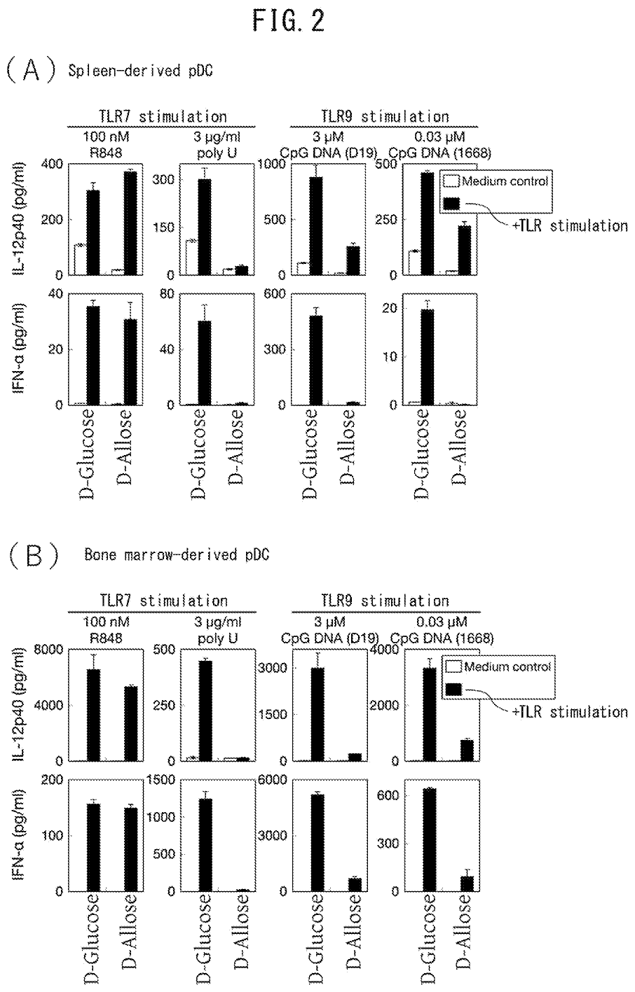

When single-stranded RNA of Toll-like receptor 7 (TLR7) ligand (poly(U)) or CpG DNA of TLR9 ligand (D19 and 1668) were used for stimulation of pDCs, interferon-α (IFN-α) and interleukin-12 (IL-12) were produced in the culture supernatant. When D-allose was included in the medium instead of D-glucose, cytokine production accompanying the stimulation was found to be notably attenuated (FIG. 2). When D-allulose, L-allulose, D-glucose or D-fructose was used, such attenuation of cytokine production as with D-allose was not observed.

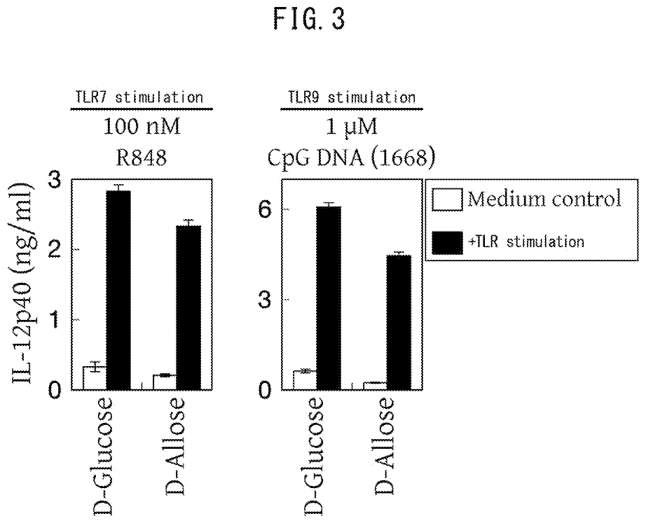

The cDC subset of dendritic cells having different function produce IL-12 after CpG DNA (1668) stimulation, but the IL-12 production was not lowered even when D-allose was included in the medium (FIG. 3). Macrophages in the intraperitoneal region of mice produce TNF-α upon lipopolysaccharide (LPS) stimulation, simultaneously producing nitric oxide, and this response was not attenuated whether including D-allose, D-allulose, L-allulose or D-fructose in the medium (not shown).

(2-2) D-Allose Suppresses Cytokine Production by Plasmacytoid Dendritic Cells after High-Molecular-Weight TLR7 Ligand Stimulation, but does not Suppress Cytokine Production after Low-Molecular-Weight TLR7 Ligand Stimulation

The average molecular weight of the TLR7 ligand poly(U) is about 800,000 or greater (by electrophoresis). Several low molecular weight forms of TLR7 ligand exist, representative of which is the imidazoquinoline compound R848 (Resiquimod) with a molecular weight of 314.38. When low molecular weight TLR7 ligands including R848 were used to stimulate pDCs, production of IFN-α and IL-12 was not reduced even in the presence of D-allose (FIG. 2). The results obtained differed from those with poly(U) stimulation.

After activating pDCs or cDCs in medium containing D-allose, the cytokines produced were quantified. As a result, pDC cytokine production was lowered while cDC cytokine production was not lowered, indicating that D-allose only suppresses pDC function.

1. A cytokine production suppressing composition containing D-allose as the active ingredient.

2. The composition according to claim 1, which suppresses production of a cytokine by plasmacytoid dendritic cells.

3. The composition according to claim 1 or 2, wherein the cytokine is type I interferon.

4. The composition according to any one of claims 1 to 3, wherein the D-allose is D-allose and/or a derivative thereof, and/or a mixture of the same.

5. The composition according to claim 4, wherein the D-allose derivative is one or more D-allose derivatives selected from the group consisting of sugar alcohols in which a carbonyl group of the D-allose is an alcohol group; uronic acids in which an alcohol group of the D-allose is oxidized; amino sugars in which an alcohol group of the D-allose is replaced with an amino group; and D-allose derivatives in which any hydroxyl group of the D-allose is replaced with a hydrogen atom, halogen atom or an amino, carboxyl, nitro, cyano, lower alkyl, lower alkoxy, lower alkanoyl, lower alkanoyloxy, lower alkoxycarbonyl, mono- or di-lower alkyl substituted amino, aralkyl, aryl or heteroaryl group.

6. The composition according to any one of claims 1 to 5, which is for treatment or prevention of a disease associated with cytokine overproduction.

7. The composition according to any one of claims 1 to 6, which is a drug.

8. The composition according to any one of claims 1 to 6, which is a food.

9. The composition according to claim 8, wherein the food is a health functional food or dietary supplement.

10. The composition according to claim 9, wherein the health functional food is a specified health food or nutritional function food.

11. A method for treatment or prevention of a disease associated with cytokine overproduction in a subject, comprising:
administering a cytokine production suppressing composition containing D-allose as the active ingredient, to the subject in need thereof.

12. The method according to claim 11, which suppresses production of cytokines by plasmacytoid dendritic cells.

13. The method according to claim 11 or 12, wherein the cytokine is type I interferon.

14. The method according to any one of claims 11 to 13, wherein the D-allose is D-allose and/or a derivative thereof, and/or a mixture of the same.

15. The method according to claim 14, wherein the D-allose derivative is one or more D-allose derivatives selected from the group consisting of sugar alcohols in which a carbonyl group of the D-allose is an alcohol group; uronic acids in which an alcohol group of the D-allose is oxidized; amino sugars in which an alcohol group of the D-allose is replaced with an amino group; and D-allose derivatives in which any hydroxyl group of the D-allose is replaced with a hydrogen atom, halogen atom or an amino, carboxyl, nitro, cyano, lower alkyl, lower alkoxy, lower alkanoyl, lower alkanoyloxy, lower alkoxycarbonyl, mono- or di-lower alkyl substituted amino, aralkyl, aryl or heteroaryl group.

16. The method according to any one of claims 11 to 15, wherein the composition is administered as a drug.

17. The method according to any one of claims 11 to 15, wherein the composition is administered as a food.

18. The method according to claim 17, wherein the food is a health functional food or dietary supplement.

19. The method according to claim 18, wherein the health functional food is a specified health food or nutritional function food.
